(19) United States
(12) Patent Application Publication
(10) Pub. No.: US 2015/0144575 A1
(43) Pub. Date: May 28, 2015

(54) UVA GERMICIDAL DEVICE

(71) Applicant: UVCleaning Systems, Inc., Beaverton, OR (US)

(72) Inventor: R. Thomas Hawkins, II, Aloha, OR (US)

(21) Appl. No.: 14/402,673

(22) PCT Filed: Mar. 12, 2013
(86) PCT No.: PCT/US2013/030387

§ 371 Date: Nov. 20, 2014

Related U.S. Application Data
(60) Provisional application No. 61/649,823, filed on May 21, 2012.

(51) Int. Cl. C02F 1/32; A61L 2/10

(52) U.S. Cl. CPC C02F 1/325; A61L 2/10; C02F 2201/3222; C02F 2303/04
USPC 210/748.11; 250/432 R

(57) ABSTRACT

Disclosed fluid purification systems can comprise a treatment vessel configured to contain a fluid to be purified, a source of ultraviolet radiation with at least 50% of its emitted spectral energy at wavelengths between 315 nm and 400 nm positioned exterior to the treatment vessel, and a portion of a wall of said treatment vessel that is substantially transparent to the ultraviolet radiation emitted by the source of ultraviolet radiation, located so that at least 50% of the ultraviolet radiation propagates through the substantially transparent portion of the wall and is configured to propagate into fluid to be treated within the vessel for the purpose of killing or disabling pathogenic microorganisms in the fluid.

### CROSS-REFERENCE TO RELATED APPLICATION

This application claims the benefit of U.S. Provisional Patent Application No. 61/649,823, entitled “UVA GERMICIDAL DEVICE” and filed on May 21, 2012, which is incorporated by reference herein in its entirety.

### FIELD

This application relates to methods and devices for disinfecting a fluid, such as water, using ultraviolet radiation.

### BACKGROUND

U.S. Pat. No. 1,196,481, issued Aug. 29, 1916, describes using UV light from a low-pressure mercury lamp for disinfection of water. As is generally accepted in the art, the key to disinfection of water using light is to illuminate the water with sufficient optical energy flux to kill or deactivate live microorganisms in the water. The optical energy flux required for disinfection is dependent on the spectrum of the light source and is generally measured in units of joules per square centimeter (J/cm²). This energy flux is the product of the irradiation intensity in watts per square centimeter (W/cm²) and the irradiation time in seconds, so that improving water throughput in an optical disinfection system requires reducing the required energy flux, increasing optical intensity or increasing irradiation time.

Nucleic acid absorption is well known to peak at approximately 265 nm, declining at longer wavelengths and down by more than a factor of ten at 300 nm. As a result, gas discharge lamps with significant spectral energy at wavelengths below 280 nm were preferred sources for UV disinfection through the 20^{th} century. Cylindrical geometries of such sources, together with the lack of high efficiency reflectors at wavelengths below 280 nm, drove UV disinfection systems to depend on radial optical designs, wherein UV light emitted radially outward from the cylindrical source propagates through fluid outside the source. Radial emission intensity from a cylindrical source diminishes with the square of the distance from the source, typically limiting utilization of the UV from the source to a path length that is well below the UV absorption path length in low-turbidity water. Furthermore, these properties of cylindrical sources mean that a germicidal discharge lamp must be immersed in the water flow path in a practical implementation, requiring a window to protect the lamp from the water and producing issues common to all such systems—including contaminant deposition onto the protective window, the possibility of discharge lamp breakage with resulting mercury contamination of the water, and heating of the fluid by the non-germicidal portion of lamp emission spectra. The disinfection irradiation time in such a cylindrical geometry is limited to the dwell time of the water in passing the cylindrical source. Various methods have been discovered for increasing effective dwell time. For example U.S. Pat. No. 4,255,383, issued Mar. 10, 1981, describes recirculating water through a UV water disinfection system to increase the irradiation time, and U.S. Pat. No. 6,099,799, issued Aug. 8, 2000, describes use of concentric water flow chambers plumbed in series to increase the irradiation time, although this geometry does not significantly improve water throughput.

UVC LEDs (peak emission wavelengths below 280 nm) produce low (˜mW maximum) continuous output power per emitter, at low (<1%) electrical conversion efficiency and with short (<1000 hours) useable lifetimes.

These properties of low electrical conversion efficiency, low output power and short lifetime for existing UVC LEDs remain as obstacles to successful implementation of an LED-based photodisinfection system. As a result, there exists a need to identify more reliable germicidal LED sources, coupled with system designs that efficiently use the emission from available LED sources to produce increased irradiation intensities as well as increased effective irradiation times that meet water treatment throughput requirements.

### SUMMARY

Disclosed are apparatuses and methods for disinfecting a fluid, such as water, using wavelengths significantly longer than those previously considered germicidal. Wavelengths below 300 nm are well-known to be capable of killing and/or sterilizing microorganisms such as bacteria, viruses and protozoa/cysts, with peak efficacy observed at approximately 265 nm. Ultraviolet radiation at longer wavelengths is absorbed only weakly by microbial entities and has therefore not been considered viable for photodisinfection applications.

Commercially available single-emitter LEDs with peak wavelengths in the 350-400 nm range can produce watts of UV output power with efficiencies >15%. In addition, the optical brightness (optical output power per unit emitting area per unit emitting angle) of these available UVA LED sources is much greater than for a discharge lamp, allowing efficient optical coupling of such light into a treatment vessel. Still further, at UVA wavelengths greater than 315 nm, and especially greater than 350 nm, practical materials that reflect or transmit light include a variety of glasses and polymer materials, and the number of these materials is much greater than those useable below 300 nm. Such materials enable optical designs that can effectively contain light within a treatment vessel so that light intensity I at distance R from a light source diminishes more slowly than the typical 1/R² intensity decline observed with point or cylindrical light sources. This improves intensity throughout a treatment vessel and allows the treatment vessel to be extended in at least one dimension, increasing treatment volume within the vessel. For example, an extended tubular treatment vessel with a polygonal, elliptical, circular or other cross section can be used to increase dwell times and treatment efficacy. Furthermore, reflective end surfaces in such a treatment vessel can allow light to make multiple round trips within the vessel, raising the intensity within the chamber beyond the intensity achievable with a UVA LED source alone. In the case of treating low-turbidity water, the UVA treatment vessel length and the resulting irradiation dwell time in the treatment vessel are limited fundamentally by the optical absorbance of the water itself. UV-visible optical transmission by pure water is greatest near 400 nm, and is 5-10 times greater in the 350-400 nm portion of the UVA spectrum than in the 250-280 nm range generally considered germicidal. This transmission difference increases with addition of impurities such as salts and organic substances to the water, so that UVA germicidal treatment vessels can be significantly longer than UVC treatment vessels. As a result of these considerations, both irradiation intensities and irradiation times can be larger in the UVA than in the UVC, resulting in UVA germicidal energy fluxes large enough to overcome the efficacy disadvantage arising from the lower UVA germicidal efficiency at the longer wavelengths. Furthermore, UVA radiation is less photochemically active than UVC, reducing the rate at which photolytic processes deposit contaminants from the treated fluid onto the windows and walls of the treatment vessel, and thereby further increasing the reliability of a UVA germicidal system compared with a UVC germicidal system. Still further, lower-cost silicon photodetectors can monitor UVA flux in a treatment vessel through lower-cost windows in vessel walls than are practical with UVC germicidal irradiation technologies.

Embodiments can comprise a treatment vessel with one or more UV LEDs or other compact UV light emitters that irradiate fluid within the vessel through at least one window in the vessel in order to disinfect the fluid. The treatment vessel can comprise a chamber filled with fluid that is treated by the UV irradiation in a batch process. Alternately, the vessel can contain at least one inlet and one outlet so that fluid enters into one portion of the vessel and exits from another portion of the vessel, and so that the UV irradiation acts to disinfect the fluid during its dwell time within the vessel. Desirably, the treatment vessel comprises at least one portion that is tubular in shape, with a length along one dimension greater than its maximum width transverse to the length dimension, and with the light from the one or more compact UV sources directed substantially along this length dimension within this tubular portion of the vessel. Further embodiments can include reflective features on or around this tubular portion of the treatment vessel that constrain ultraviolet radiation directed into the fluid to travel substantially along a length of fluid to be treated, especially within a transverse dimension that is substantially less than this length, and with substantially homogeneous intensity across any cross-section of the treatment length. Still further embodiments can include reflective surfaces at each end of the fluid treatment vessel that can return a fraction of the light propagating along the vessel back into the vessel. By reflecting a significant fraction of the light propagating along this fluid length from each end of the vessel, the flux of light within the fluid is increased, and multiple reflections further increase the flux. Reflecting a fraction ρ of the light incident on each end of the treatment vessel back into the vessel increases the flux of light within the vessel by a factor of up to 1/(1−ρ). For example, a 50% reflection at each end of the treatment vessel can double the effective flux within the vessel. Homogeneity of flux within the liquid, together with increased intensity due to multiple reflections along the length of the illuminated fluid region, can result in increased dose during the contact time of the light with the flowing fluid. Irradiating a fluid substantially along the axis of a tubular treatment vessel, optimizing UV dose through improved optical design, and/or using efficient, high-power UVA LEDs, can render UVA photodisinfection of fluids, including water, practical. In addition, methods for monitoring UVA flux within the treatment vessel are disclosed herein, as can be desirable for assuring system performance.

In a first embodiment, fluid is placed inside an elongated treatment vessel. Ultraviolet radiation from one or more UVA sources enters the fluid through a window or opening in the exterior wall at one end of the elongated vessel and travels along the long axis of the vessel. Each photon, or quantum of UV light, has the opportunity to interact with microbial contaminants along the entire length of the treatment vessel, and the intensity of light at any point within the vessel is inversely proportional to the cross-sectional area of the vessel at that point. For a low-turbidity, UVA-transmitting fluid in the vessel, the quantum efficiency of the disinfection process is approximately proportional to the length of the treatment vessel.

In a second embodiment, the UVA radiation traveling along the long axis of the elongated treatment vessel is reflected by reflective interior surfaces of the vessel walls (such as polished or coated stainless steel), or by a coating or other reflective material outside substantially transparent vessel walls. The UVA radiation intensity can be substantially homogenized across the interior of the treatment vessel as the radiation propagates through the fluid, and reduced loss of light at the side walls of the vessel results in higher UVA radiation intensity along the length of the vessel.

In a third embodiment, one or both end surfaces of the treatment vessel, except for one or more windows large enough to admit the UV from the UVA source or sources, can also be reflective to recirculate the UV radiation back and forth within the treatment vessel and thereby to maximize the UV irradiation flux over the treatment volume. The length of the treatment vessel can be set at the largest practical length to maximize treatment volume within a practical irradiation time. The largest practical length can be determined by optical losses due to absorption of the light by the fluid, by contaminants in the fluid, and/or by the walls of the treatment vessel.

In a fourth embodiment, one or more photodetectors, such as silicon photodiodes or other devices, can be attached to or embedded in treatment vessel walls in order to monitor the flux of UVA radiation within the treatment vessel. Use of window materials and photodetectors suitable for UVA radiation applications allows greater design freedom and lower costs than are practical at the UVC wavelengths traditionally preferred for UV disinfection.

In a fifth embodiment, fluid enters at or near one end of the elongated treatment vessel, flows along the length of the vessel and exits at or near the other end. Ultraviolet radiation from one or more UVA LEDs enters the fluid at one end of the vessel and is transmitted through the fluid along the length of the treatment vessel. Disinfection by the UVA radiation is proportional to the intensity of the radiation and to the dwell time of the flowing fluid within the irradiated portion of the treatment vessel. Making the interior surfaces of the vessel walls reflective—for example by using polished or coated stainless steel walls or by using substantially transparent vessel walls and coating or otherwise adding reflective material to the exterior of the walls—increases the UVA intensity within the fluid and thereby improves disinfection performance. Making the end surfaces of the treatment vessel reflective, except for a window large enough to admit the UVA from the LED or other compact UVA source, recirculates the UV radiation back and forth within the treatment vessel and thereby further increases the UV irradiation flux over the treatment volume. Again, the length of the treatment vessel can be set at the largest practical length to maximize the irradiation time as constrained by absorbance of the fluid and its flow rate through the vessel.

In a sixth embodiment, one or more LEDs or other compact UVA sources can be mounted at each end of a UVA-reflective treatment vessel, to increase the optical flux within the vessel and to improve uniformity of radiation intensity along the length of the treatment vessel.

In a seventh embodiment, the treatment vessel can be shaped or formed at one end or at both ends in order to guide light within the vessel and to reduce the extent of under-illuminated regions in the liquid near a window illuminated by one or more LED or other compact UVA sources outside the vessel. One possible profile of such a shaped or formed vessel end can approximate a parabolic, ellipsoidal or spherical profile. Another possible profile of such a shaped or formed vessel end can be a tapered region, such as a taper with a half-angle of approximately 30-45 degrees, about the axis of the treatment vessel. In addition to guiding light to reduce the extent of under-illuminated regions within the vessel, such a shaped or formed vessel end may also serve to increase reflectivity at the end of the treatment vessel. The detailed profile of such a shaped region can be designed with optical modeling tools to optimize the homogeneity of UV radiation intensity throughout the treatment vessel for the specific emission profile of the one or more UV emitters employed.

In an eighth embodiment, an optical element can be inserted between one or more UVA LEDs or other compact UVA sources and the corresponding window or windows into the treatment vessel. This optical element may incorporate either refractive or reflective features, or both, in order to improve homogeneity of ultraviolet radiation within the treatment vessel, and may also improve optical coupling through at least one window into the treatment vessel.

In a ninth embodiment, an optical element can be formed by or integrated into a window at either or both ends of the treatment vessel. Again, this optical element may serve to improve homogeneity of ultraviolet radiation within the treatment vessel, and may also improve optical coupling through a window into the treatment vessel.

Additional embodiments can be implemented without departing from the spirit or the scope of this disclosure. For example, other combinations of refractive and reflective optical elements can be used to achieve substantially uniform UVA intensity throughout the treatment vessel.

The foregoing and other objects, features, and advantages of the disclosure will become more apparent from the following detailed description, which proceeds with reference to the accompanying figures.

### BRIEF DESCRIPTION OF THE DRAWINGS

FIG. 1 shows an embodiment of a UVA fluid disinfection module comprising a treatment vessel open at one end with a UVA source illuminating fluid through an opening.

FIG. 2 shows another embodiment of a UVA fluid disinfection module comprising a treatment vessel with a UVA source in a cap at a top end and positioned to illuminate fluid in the vessel. Side walls of the treatment vessel have UVA reflective surfaces.

FIG. 3 shows another embodiment of a UVA fluid disinfection module comprising a treatment vessel with a window at one end and a cap at opposite end. A UVA source positioned outside the window irradiates fluid within vessel through the window. Treatment vessel surfaces are UVA reflective except for the window region transmitting UVA radiation from source.

FIG. 4 shows another embodiment of a UVA fluid disinfection module comprising a UVA source illuminating fluid through a window on or in a cover of the treatment vessel opening, and UVA reflective surfaces on or around the vessel.

FIG. 5 shows another embodiment of a UVA fluid disinfection module comprising a UVA source illuminating fluid through a window, UV reflective surfaces on or around the treatment vessel, and one or more photodetectors mounted exterior to or within walls of the treatment vessel to monitor UV flux within the treatment vessel.

FIG. 6 shows another embodiment of a UVA fluid disinfection module comprising a flow-through treatment vessel with a window at one end and a UVA source illuminating fluid within the vessel through the window.

FIG. 7 shows another embodiment of a UVA fluid disinfection module comprising a flow-through treatment vessel with a window at one end and a UVA source illuminating fluid within the vessel through the window. Walls of the treatment vessel have UVA reflective surfaces.

FIG. 8 shows another embodiment of a UVA fluid disinfection module comprising a flow-through treatment vessel with a window at one end and a UVA source illuminating fluid within the vessel through the window. Walls and ends of the treatment vessel have UVA reflective surfaces.

FIG. 9 shows another embodiment of a UVA fluid disinfection module comprising a flow-through treatment vessel with a window at both ends, UVA sources irradiating fluid through the windows, and UVA reflective surfaces on or around vessel walls and ends.

FIG. 10 shows another embodiment of a UVA fluid disinfection module comprising a flow-through treatment vessel with windows at both ends, a UVA LED source illuminating fluid through one of the windows, UVA reflective surfaces on or around the vessel, and monitor photodetectors on at least one window.

FIG. 11 shows another embodiment of a UVA fluid disinfection module comprising a UVA source illuminating fluid through a window, UV reflective surfaces on or around the treatment vessel, and a transition zone at an illuminated end of vessel to guide and homogenize UV radiation intensity over the cross section of the treatment vessel.

FIG. 12 shows another embodiment of a UVA fluid disinfection module comprising a flow-through treatment vessel with a tapered profile, windows at both ends, UVA sources illuminating fluid through the windows, and UVA reflective surfaces on or around the vessel.

FIG. 13 shows another embodiment of a UVA fluid disinfection module comprising a flow-through treatment vessel with windows at each end, UVA sources illuminating fluid through optical elements exterior to the windows, and UVA reflective surfaces on or around the vessel.

FIG. 14 shows another embodiment of a UVA fluid disinfection module comprising a flow-through treatment vessel, a UVA source illuminating fluid through a combined collimating lens and window at one end of the vessel, and UVA reflective surfaces on or around the vessel.

FIG. 15 shows another embodiment of a UVA fluid disinfection module comprising a flow-through treatment vessel with a window at a tapered end, a UVA source illuminating fluid through an optical element integrated into the window, and UVA reflective surfaces on or around the vessel.

FIG. 16 is a plot of UV dose required to kill E. coli as a function of illumination wavelength, together with modeled disinfection performance by two embodiments of the disclosed UVA disinfection systems at 1000 gallons per day fluid throughput rate. One modeled fluid treatment system embodiment is substantially as shown in FIG. 8, with a 10 W UVA LED having Lambertian emission at one end of a cylindrical treatment vessel 5 cm in diameter and 100 cm long. A second modeled fluid system embodiment is substantially as shown in FIG. 14, with a 10 W UVA LED with Lambertian emission at one end of a cylindrical treatment vessel 5 cm in diameter and 100 cm long and with a collimating optic between the LED emitter and the cylindrical treatment vessel.

FIG. 17 is a graph showing UV dose as a function of reflectivity of cylinder inner surface for a cylindrical UVA fluid treatment system substantially as shown in FIG. 8, with a 10W Lambertian emitter at one end of a cylinder having an inner diameter D=5 cm, and treatment length L=200, 100 and 50 cm. The graph demonstrates that increasing treatment length (equivalent to reducing the diameter to length ratio D/L) increases the UV dosage at a fixed fluid throughput rate, for any practical reflectivity of the cylinder walls. Note that this model assumes no reflection by the end of the treatment vessel opposite the UVA source end.

FIG. 18 is a graph showing UV dose as a function of reflectivity of cylinder inner surface for a cylindrical UVA fluid treatment system substantially as shown in FIG. 14, with a 10W Lambertian emitter and collimating optical element at one end of a cylinder having inner diameter D=5 cm, and treatment length L=200, 100 and 50 cm. The graph demonstrates that increasing treatment length (equivalent to reducing the diameter to length ratio D/L) increases the UV dosage at a fixed fluid throughput rate, for any practical reflectivity of the cylinder walls. The model also demonstrates that UVA dose is significantly improved by the use of a collimating optical element (cf FIG. 17). Note that this model assumes no reflection by the end of the treatment vessel opposite the UVA source end.

### DETAILED DESCRIPTION

The following is a list of major drawing elements in numerical order:
110 Fluid treatment vessel
120 Fluid within treatment vessel
130 Cap (removable cover) sealing end of treatment vessel
140 Window in fluid fill port cover
150 Fluid inlet port
160 Fluid outlet port
190 Portion of fluid treatment vessel configured to optimize optical performance
220 Reflective material or coating
240 UVA LED light source
250 Window in wall of fluid treatment vessel
260 Photodetector
280 Optical element external to fluid treatment vessel
290 Optical element in exterior surface of fluid treatment vessel

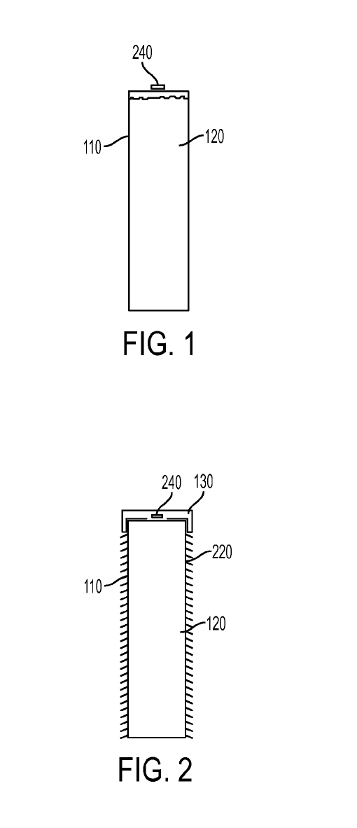

Referring first to FIG. 1, the basic construction of a UVA fluid disinfecting device in accordance with a first embodiment is shown. An elongated treatment vessel 110 containing fluid 120 is irradiated by UV light from UVA source 240 positioned exterior to the treatment vessel. The cross section of the treatment vessel 110 can have a substantially elliptical, circular, polygonal or other cross section profile. The length of the treatment vessel 110 is desirably greater than the maximum width of the vessel.

Referring next to FIG. 2, the basic construction of a UVA fluid disinfecting device in accordance with a second embodiment is shown. An elongated treatment vessel 110 containing fluid 120 is irradiated by UV light from UVA source 240 positioned at or near one end of the treatment vessel. At least one reflective material 220 covers the side walls of the treatment vessel. The cross section of the treatment vessel is desirably optimized to homogenize the UV radiation intensity across the cross section within a minimum distance along the treatment vessel. The length of the treatment vessel 110 is desirably greater than the maximum width of the vessel, so that the UV radiation intensity is substantially uniform across the cross section of the vessel over the majority of the length of the vessel. The cross section profile and area of the treatment vessel are desirably substantially constant along the length of the UV illuminated region. FIG. 2 also demonstrates that the UVA source 240 can be mounted within and/or on a cap 130 covering the end of the vessel.

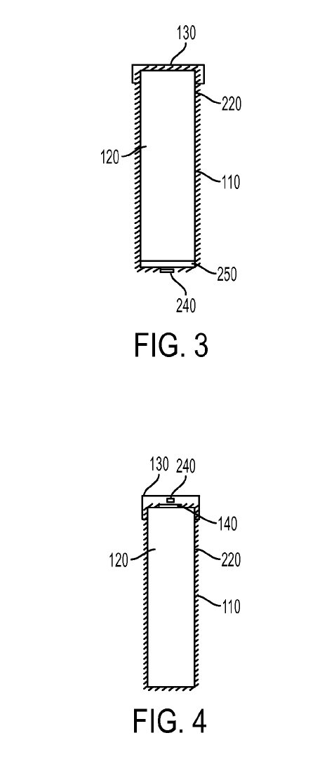

Referring next to FIG. 3, the basic construction of a UVA fluid disinfecting device in accordance with a third embodiment is shown. An elongated treatment vessel 110 containing fluid 120 is irradiated by UV light from UVA source 240 positioned exterior to a window 250 at one end of the treatment vessel. At least one reflective material 220 covers substantially all of the interior or exterior surfaces of the treatment vessel 110 as well as the inner (fluid contacting) surface of the cap 130 covering the end of the vessel opposite the UVA source, except for the portion of the window 250 through which the UVA radiation enters the vessel. The window 250 can comprise a substantially transparent optical element sealed onto or into an exterior wall of the treatment vessel or, if the exterior wall of the treatment vessel is made of a substantially UVA-transparent material (e.g., a glass such as Schott BK-7 or a polymer such as acrylic), this window may simply be a region of a the exterior wall that is not coated or otherwise covered by a reflective material. Reflecting the UVA light back down the length of the treatment vessel further increases the intensity within the fluid. If no light were reflected from the ends of the treatment vessel, the ultraviolet intensity I₀ at any given point within the treatment vessel is approximately the sum of the powers from each of the UV sources divided by the cross-section area at that point. For a mean treatment vessel round trip ultraviolet reflectivity ρ², corresponding to the fraction of ultraviolet power making a round trip through the vessel from any internal starting point within the vessel to one end of the vessel, reflected from that end back along the vessel through the starting point to the other end of the vessel and then reflected back to the starting point, the mean intensity in the treatment vessel is approximately

I=I₀·(1+ρ+ρ²+ρ³+ρ⁴+ . . . )=I₀/(1−ρ). (D-2)

This increase in effective ultraviolet intensity is optimized by making the effective round trip reflectivity ρ² as large as possible. Use of UVA radiation allows significantly higher UV intensities within the treatment vessel than in a UVC germicidal treatment vessel, because a) UVA LED sources are significantly more powerful than UVC LED sources, b) Material reflectivities are generally higher in the UVA spectral region than in the UVC, and c) UVC absorption in fluids, including water, is generally higher than UVA absorption.

The germicidal UV flux dose F_{K}(λ) required to kill or disable microorganisms in a fluid is wavelength dependent and results in a minimum treatment time τ_{K}(λ) at intensity I given by

τ_{K}(λ)≧F_{K}(λ)/I. (D-3)

In order to assure all microorganisms are killed or disabled by the treatment system, this minimum dwell time can satisfy the relationship

τ_{Dwell}≧F_{K}(λ)/I. (D-4)

Through use of reflective materials around or on the exterior surfaces of the treatment vessel, irradiation intensity is increased within the vessel and the length of the treatment vessel can be determined by the absorbance of the fluid being treated.

FIG. 4 also shows a UVA fluid disinfecting device in accordance with the third embodiment. The treatment vessel can have a fixed or removable cap 130 incorporating a window 140 and a UVA source 240 that irradiates the fluid 120 within the treatment vessel 110 through the window 140.

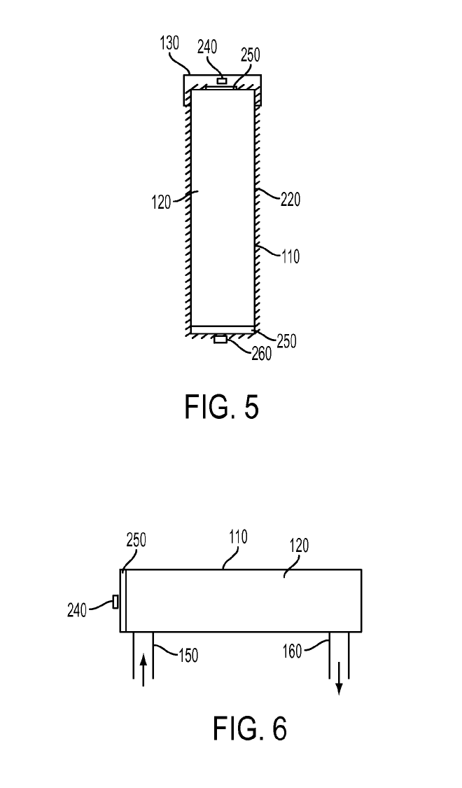

Referring next to FIG. 5, the basic construction of a UVA fluid disinfecting device in accordance with a fourth embodiment is shown. UVA source 240 one end of the vessel irradiates the fluid 120 within the elongated treatment vessel 110 through windows 250. In addition, a UVA photodetector 260 is positioned outside another window to monitor UVA intensity transmitted through the fluid in the vessel. Such monitoring enables sensing loss of ultraviolet power from one or more UVA LED sources as well as growth of films on the interior surfaces of the treatment vessel that result in an internal loss of ultraviolet power. Photodetectors sensitive to UVA wavelengths produced by the UVA sources are readily available commercially and are significantly less expensive than UVC photodetectors.

Referring next to FIG. 6, the basic construction of a flow-through UVA fluid disinfecting device in accordance with a fifth embodiment is shown. The treatment vessel can be configured as a flow-through device by the addition of at least one inlet port 150 at one end of the vessel and at least one outlet port 160 at the other end of the vessel. A fluid to be treated, such as water, enters input port 150, flows along the length of the treatment vessel 110, and flows out of the vessel through outlet port 160. The inlet port and/or the outlet port can be fabricated of the same material as the remainder of the treatment vessel, or made of another material and attached to the body of the treatment vessel. At least one UVA LED or other compact UVA source is positioned external to the vessel so that the UVA light emitted by the source passes through a window 250 in an exterior wall of the treatment vessel, irradiating the fluid during its dwell time within the vessel. In this flow-through apparatus, the treatment time is the minimum dwell time τ_{Dwell} of a microorganism in the treatment vessel determined by the flow characteristics of the fluid into, through and out of the treatment vessel, and by the flow rate (e.g., liters per second) of the fluid through the vessel.

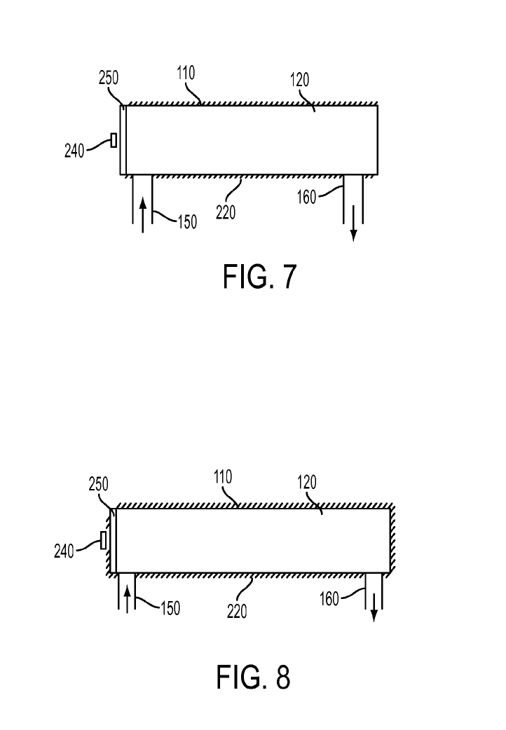

Referring next to FIG. 7, reflective material 220 within or on the walls of treatment vessel 110 can be added to the vessel. This reflective material can comprise the interior polished surface of a metal (e.g., stainless steel) vessel, a reflective layer (e.g., a metal or multilayer coating) attached to the interior surface of the vessel walls, a reflective layer (e.g., a metal or multilayer coating) attached to the exterior surface of substantially transparent vessel walls, a reflective material (e.g., a metal, polymer or other foil) wrapped or otherwise mounted on the exterior of a substantially transparent vessel walls, or any combination of these and/or other reflective technologies. Because of the reflectivity of the walls of the treatment vessel, UVA power diverging from the source is reflected back into the fluid within the vessel rather than being lost to absorption by the walls. In addition, this reflected energy tends to homogenize the UVA intensity over the cross section of the vessel.

Referring next to FIG. 8, reflective material can also be added to the end surfaces of the treatment vessel. The openings in the treatment vessel wall through which the inlet and outlet flows pass cannot typically be made reflective, so the diameter of these openings can be as small as possible while accommodating the fluid throughput required of the apparatus, in order to maximize the UV intensity I within the treatment vessel.

For a flow rate R_{FLOW} (in cm³ per second) through a vessel with cross-sectional area A_{V} (in cm²) and length L_{V} (in cm), the dwell time in the treatment vessel is approximately

τ_{Dwell}=α·A_{V}·L_{V}/R_{FLOW}≧F_{K}(λ)/I, (D-5)

where α<1 is a correction factor depending on the flow characteristics of the treatment vessel. Equation D-5 quantifies the advantage of both increased treatment vessel length in maximizing irradiation dwell time within the treatment vessel length and increased UV intensity I in minimizing dwell time required at flow rate R_{FLOW}.

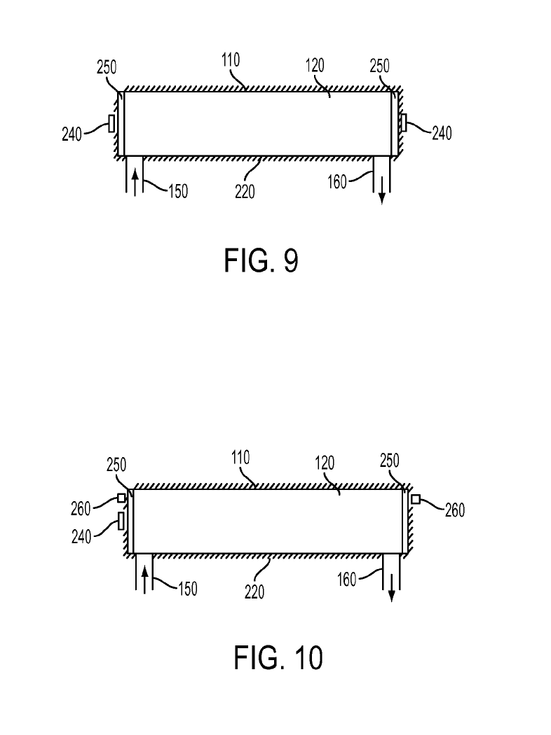

Referring next to FIG. 9, the basic construction of a flow-through UVA fluid disinfecting device in accordance with a sixth embodiment is shown. One or more UVA LED sources 240 can be mounted outside a window 250 at each end of the treatment vessel 110 in order to further increase the ultraviolet intensity within the treatment vessel and thereby reduce the required treatment time within the vessel. FIG. 10 illustrates the use of photodetectors at both ends of the treatment vessel in order to improve monitoring of UVA intensity throughout the vessel.

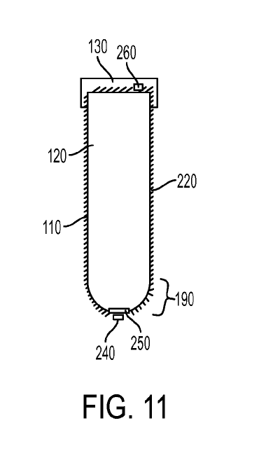

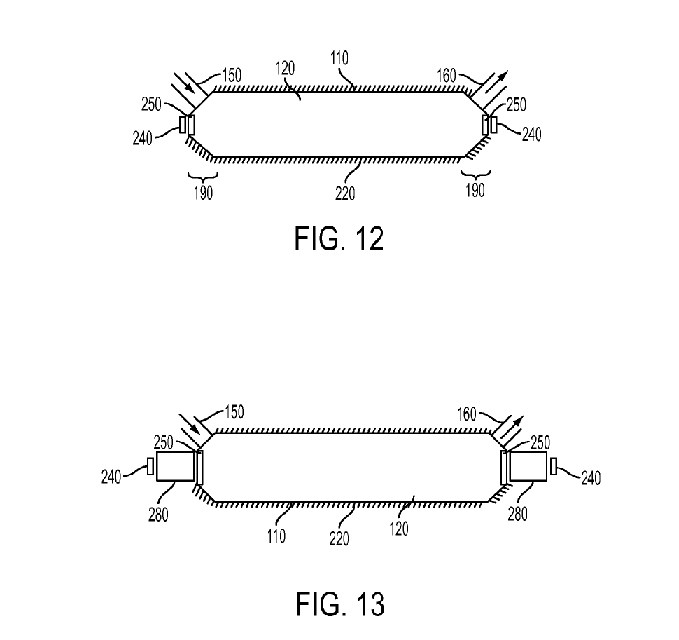

Referring next to FIG. 11, the use of a shaped transition zone 190 at the UV input end of a treatment vessel is shown in accordance with a seventh embodiment. Divergent light from a UVA source 240 coupled through a window 250 can be fully or partially collimated through reflection from the end surfaces in the shaped transition zone of the treatment vessel in order to reduce optical loss within the treatment vessel. The detailed profile of such a transition region can be configured to optimize performance with the spatial emission profile of the corresponding UVA LED source. FIG. 12 shows another vessel in accordance with this embodiment, wherein UVA sources 240 at each end of a treatment vessel are at least partially collimated by the tapered transition zones 190.

FIG. 13 illustrates schematically the use of at least one external optical element 280 mounted between a UVA source 240 and the corresponding window of the treatment vessel 110, in accordance with an eighth embodiment. One advantage of such an optical element can be controlling the size and divergence of the virtual UVA source at the output of the optical element to rapidly achieve uniform distribution of intensity across the treatment vessel cross section. Another advantage of such an optical element can be to improve coupling of light from the UVA source into the treatment vessel.

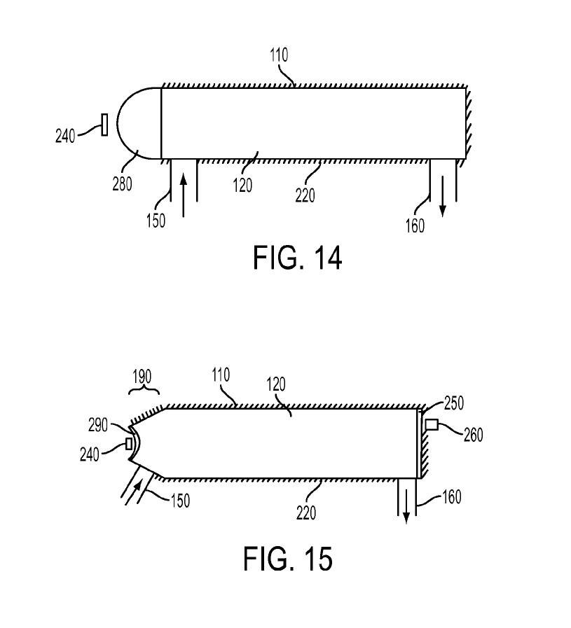

Referring next to FIG. 14, a collimating optical element 280 can be integrated into the end of a treatment vessel 110, in accordance with a ninth embodiment, to improve optical coupling of light from an optical source 240, such as an LED with ˜Lambertian spatial emission, into the vessel.

FIG. 15 shows schematically how an integrated optical element 290 can be combined with a shaped transition zone 190 at one or both ends of a treatment vessel 110 to improve optical coupling and homogeneity within the vessel.

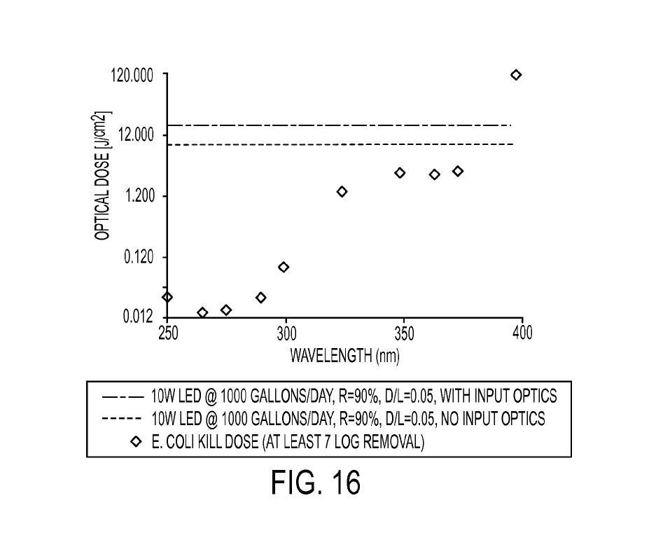

Turning now to FIG. 16, a graph shows the dependence of the measured “kill dose” required for E. coli bacteria in water on ultraviolet wavelength, over the 250-400 nm wavelength range. This graph illustrates the more than 100× difference in UV flux required to kill E. coli between the generally accepted optimum wavelengths in the UVC range near 265 nm, and the more accessible UVA wavelengths >350 nm. Also shown in this figure is the calculated dose accessible with a single 10 W Lambertian source of UVA light and a cylindrical treatment vessel with a 5 cm inner diameter and a treatment length of 100 cm at a 1000 gal/day flow rate, both with and without a collimating optical element between the optical source and the treatment vessel. The model demonstrates that the UVA dose is well in excess of the required E. coli kill dose to wavelengths of at least 375 nm, both with and without the collimating optical element. Moreover, because the model does not include reflections from ends of the treatment vessel, achievable germicidal doses may be greater than that modeled.

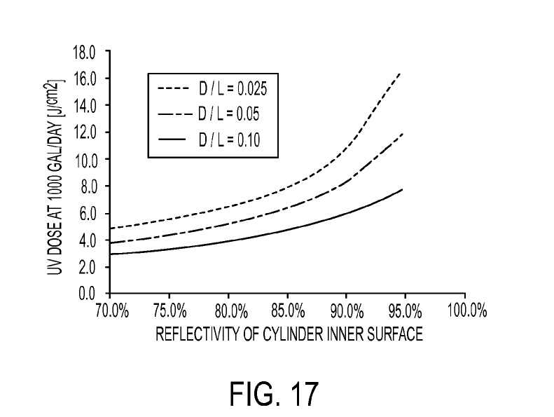

FIG. 17 shows the results of modeling a UVA fluid disinfection module like the one shown schematically in FIG. 8. The UVA source in the model is a Lambertian emitter with output power of 10 W. The cylindrical treatment vessels in this model have a 5 cm inner diameter, with lengths of 200 cm, 100 cm and 50 cm. The results illustrate the dependence of germicidal dose on reflectivity of the reflective materials either exterior to or comprising the walls of the treatment vessel. Furthermore, this figure demonstrates the germicidal dose advantage of a longer treatment vessel for any specific reflectivity of the vessel walls. Again, because the model does not include reflections from ends of the treatment vessel, the germicidal dose is expected to be greater than that modeled.

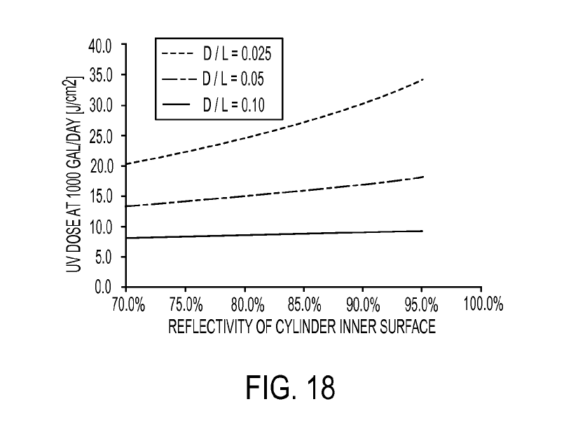

Turning next to FIG. 18, results of modeling the UVA fluid disinfection module shown schematically in FIG. 14 show the dependence of germicidal dose on reflectivity of the reflective materials either exterior to or comprising the walls of the treatment vessel. Furthermore, this figure demonstrates the germicidal dose advantage of a longer treatment vessel for any specific reflectivity of the vessel walls. Comparison with the model of FIG. 17 also shows the UVA germicidal dose advantage of using a collimating optical element for any specific wall reflectivity and treatment vessel length. Again, because the model does not include reflections from ends of the treatment vessel, the germicidal dose is expected to be greater than that modeled.

### General Considerations

For purposes of this description, certain aspects, advantages, and novel features of the embodiments of this disclosure are described herein. The disclosed methods, apparatuses, and systems should not be construed as limiting in any way. Instead, the present disclosure is directed toward all novel and nonobvious features and aspects of the various disclosed embodiments, alone and in various combinations and sub-combinations with one another. The methods, apparatuses, and systems are not limited to any specific aspect or feature or combination thereof, nor do the disclosed embodiments require that any one or more specific advantages be present or problems be solved.

Although the operations of some of the disclosed methods are described in a particular, sequential order for convenient presentation, it should be understood that this manner of description encompasses rearrangement, unless a particular ordering is required by specific language. For example, operations described sequentially may in some cases be rearranged or performed concurrently. Moreover, for the sake of simplicity, the attached figures may not show the various ways in which the disclosed methods can be used in conjunction with other methods.

As used herein, the terms “a”, “an” and “at least one” encompass one or more of the specified element. That is, if two of a particular element are present, one of these elements is also present and thus “an” element is present. The terms “a plurality of” and “plural” mean two or more of the specified element.

As used herein, the term “and/or” used between the last two of a list of elements means any one or more of the listed elements. For example, the phrase “A, B, and/or C” means “A,” “B,” “C,” “A and B,” “A and C,” “B and C” or “A, B and C.”

As used herein, the term “coupled” generally means physically coupled or linked and does not exclude the presence of intermediate elements between the coupled items absent specific contrary language.

In view of the many possible embodiments to which the principles of the disclosure may be applied, it should be recognized that the illustrated embodiments are only preferred examples and should not be taken as limiting the scope of the disclosure.

1. A fluid purification system comprising:
a treatment vessel configured to contain a fluid to be purified;
at least one source of ultraviolet radiation with at least 50% of its emitted spectral energy at wavelengths between 315 nm and 400 nm, positioned exterior to said treatment vessel; and
at least one portion of at least one wall of said treatment vessel that is substantially transparent to the ultraviolet radiation emitted by said at least one source of ultraviolet radiation, located so that at least 50% of said ultraviolet radiation propagates through said at least one substantially transparent portion of said at least one wall and is configured to propagate into fluid to be treated within the vessel for the purpose of killing or disabling pathogenic microorganisms in the fluid.

2. A fluid purification system according to claim 1, wherein the flux of said ultraviolet radiation is substantially homogeneous across the fluid flow path at each location within said treatment vessel.

3. A fluid purification system according to claim 1, wherein said treatment vessel is elongated in one dimension, and wherein the cross-section shape and size perpendicular to said elongation dimension is substantially constant at substantially all points within said treatment vessel along the elongation dimension.

4. A fluid purification system according to claim 3, wherein the length of said treatment vessel along said elongation dimension is greater than twice the largest transverse dimension of said treatment vessel at any point along said elongation dimension.

5. A fluid purification system according to claim 3, wherein at least one source of ultraviolet radiation is positioned exterior to said elongated treatment vessel at each end of the vessel.

6. A fluid purification system according to claim 1, wherein the treatment vessel incorporates at least one inlet port through which a fluid can enter the treatment vessel and at least one outlet port through which fluid can exit the treatment vessel, so that the fluid can be purified as it flows through the treatment vessel.

7. A fluid purification system according to claim 1, wherein the at least one source of ultraviolet radiation has the majority of its emitted spectral energy at wavelengths between 350 nm and 400 nm.

8. A fluid purification system according to claim 1, wherein the at least one source of ultraviolet radiation is a light emitting diode.

9. A fluid purification system according to claim 1, wherein at least a portion of the interior surfaces of the walls of said treatment vessel comprise or are coated by at least one material that substantially reflects said ultraviolet radiation.

10. A fluid purification system according to claim 1, wherein at least one portion of at least one wall of said treatment vessel substantially reflects the ultraviolet radiation from the said at least one source, thereby returning a portion of said reflected ultraviolet radiation back into the treatment vessel.

11. A fluid purification system according to claim 9, wherein the flux intensity is greater than that of a non-reflective treatment vessel due to multiple reflections of the ultraviolet radiation within the treatment vessel.

12. A fluid purification system according to claim 1, wherein at least a portion of the walls of said treatment vessel are substantially transparent to the light from the at least one source of ultraviolet radiation, and the exterior of at least a portion of said substantially transparent portion of the walls are coated or covered by at least one material that substantially reflects said ultraviolet radiation.

13. A fluid purification system according to claim 12, wherein the flux intensity is greater than two times that of a non-reflective treatment vessel due to multiple reflections of the ultraviolet radiation within the treatment vessel.

14. A fluid purification system according to claim 12, wherein at least one photo sensor is positioned outside of the at least one substantially transparent portion of the walls of said treatment vessel in order to monitor the ultraviolet radiation flux within the treatment vessel.

15. A fluid purification system according to claim 1, wherein at least one optical element is located between the at least one source of ultraviolet radiation and the at least one substantially transparent portion of the walls of said treatment vessel, said optical element serving to improve optical coupling of said ultraviolet radiation through said at least one substantially transparent portion of the walls of said treatment vessel and/or to improve homogeneity of said ultraviolet radiation within said treatment vessel.

16. A fluid purification system according to claim 1, wherein at least one optical element is incorporated into the at least one substantially transparent portion of the walls of said treatment vessel, said optical element serving to improve optical coupling of said ultraviolet radiation through said at least one substantially transparent portion of the walls of said treatment vessel and/or to improve homogeneity of said ultraviolet radiation within said treatment vessel.

17. A fluid purification system according to claim 1, wherein at least one end of said treatment vessel is shaped or formed to improve coupling of ultraviolet radiation from a source outside the treatment vessel into a substantially uniform intensity distribution over the cross section of the treatment vessel over the majority of the length of the treatment vessel.

18. A method for purifying a fluid with ultraviolet radiation, the method comprising placing the fluid into a treatment vessel wherein the fluid is illuminated by ultraviolet radiation in the UVA band of wavelengths, and continuing the illumination of the fluid for a period of time sufficient to assure all microorganisms within the fluid have been killed or deactivated.

19. A method in accordance with claim 18, wherein the fluid flows through the treatment vessel during the purification process, and wherein the dwell time of the fluid within the treatment vessel is sufficient to assure that all microorganisms within the fluid have been killed or deactivated.

20-32. (canceled)
